(19) United States
(12) Patent Application Publication
(10) Pub. No.: US 2013/0197280 A1
(43) Pub. Date: Aug. 1, 2013

(54) SYNTHESIS OF FLUORINATED OLEFINS FROM FLUORINATED ALCOHOLS

(71) Applicants: Andrei Aleksandrovich Stepanov, Moscow (RU); Victor Filippovich Cherstkov, Moscow (RU); Mario Joseph Nappa, Newark, DE (US)

(72) Inventors: Andrei Aleksandrovich Stepanov, Moscow (RU); Victor Filippovich Cherstkov, Moscow (RU); Mario Joseph Nappa, Newark, DE (US)

(73) Assignee: EI DU PONT DE NEMOURS AND COMPANY, Wilmington, DE (US)

(21) Appl. No.: 13/519,921

(22) PCT Filed: Dec. 29, 2010
(86) PCT No.: PCT/US10/62285

§ 371 Date: Sep. 24, 2012

(30) Foreign Application Priority Data
Dec. 29, 2009 (RU) 2009149445

(51) Int. Cl. C07C 17/35

(52) U.S. Cl. CPC C07C 17/35
USPC 570/142

(57) ABSTRACT

Disclosed is a process for producing a hydrofluoroalkene, R_{f}CF═CH₂ comprising contacting a hydrofluoroalkanol of structure R_{f}CF₂CH₂OH, with a lewis acid to produce a mixture, diluting said mixture with a solvent to produce a solvent mixture, contacting the solvent mixture with a reactive metal, heating the solvent mixture and reactive metal for a sufficient amount of time to produce a hydrofluoroalkene, and condensing and collecting the volatile products comprising the hydrofluoroalkene, wherein R_{f} is F, or a fluorine-substituted alkyl group.

### BACKGROUND INFORMATION

1. Field of the Disclosure

This disclosure relates in general to a process for the production of hydrofluoroalkenes, and in particular a process for the production of 2,3,3,3-tetrafluoro-1-propene, from hydrofluoroalkanols.

2. Description of the Related Art

The refrigeration industry has been working for the past few decades to find replacement refrigerants for the ozone depleting chlorofluorocarbons (CFC's) and hydrochlorofluorocarbons (HCFC's) being phased out as a result of the Montreal Protocol. The solution for most refrigerant producers has been the commercialization of hydrofluorocarbon (HFC) refrigerants. HFC's, however, are now being regulated due to concerns related to global warming.

There is always a need for new and better processes for the preparation of halocarbons that may be useful as refrigerants or in other applications such as foam expansion agents, aerosol propellants, fire suppression or extinguishing agents, solvents, and sterilants to name a few.

### SUMMARY

Disclosed is a process for producing a hydrofluoroalkene, R_{f}CF═CH₂ comprising contacting a hydrofluoroalkanol of structure R_{f}CF₂CH₂OH, with a lewis acid to produce a mixture, diluting said mixture with a solvent to produce a solvent mixture, contacting the solvent mixture with a reactive metal, heating the solvent mixture and reactive metal for a sufficient amount of time to produce a hydrofluoroalkene, and condensing and collecting the volatile products comprising the hydrofluoroalkene, wherein R_{f} is F, or a fluorine-substituted alkyl group.

The foregoing general description and the following detailed description are exemplary and explanatory only and are not restrictive of the invention, as defined in the appended claims.

### DETAILED DESCRIPTION

Disclosed is a process for producing a hydrofluoroalkene, R_{f}CF═CH₂ comprising contacting a hydrofluoroalkanol of structure R_{f}CF₂CH₂OH, with a lewis acid to produce a mixture, diluting said mixture with a solvent to produce a solvent mixture, contacting the solvent mixture with a reactive metal, heating the solvent mixture and reactive metal for a sufficient amount of time to produce a hydrofluoroalkene, and condensing and collecting the volatile products comprising the hydrofluoroalkene, wherein R_{f} is F, or a fluorine-substituted alkyl group.

In one embodiment, the group R_{f} in the above hydrofluoroalkene and hydrofluoroalkanol can be either a Fluorine atom, or a fluorine-substituted alkyl group. In one embodiment, the fluorine-substituted alkyl can be CF₃, C₂F₅, n-C₃F₇, i-C₃F₇, n-C₄F₉, or CHF₂CF₂CF₂—.

Many aspects and embodiments have been described above and are merely exemplary and not limiting. After reading this specification, skilled artisans appreciate that other aspects and embodiments are possible without departing from the scope of the invention. Other features and benefits of any one or more of the embodiments will be apparent from the following detailed description, and from the claims.

Before addressing details of embodiments described below, some terms are defined or clarified.

As used herein dehydroxyfluorinating refers to removing a hydroxyl group and a fluorine atom from adjacent carbon atoms of a hydrofluoroalkanol to form a hydrofluoroalkene.

As used herein, a Lewis acid is a chemical compound, A, that can accept a pair of electrons from a Lewis base, B, that acts as an electron-pair donor, forming an adduct, AB.

As used herein, reactive metal refers to reactive metals such as magnesium turnings, activated zinc powder, aluminum, and a powder of any of the following metals: magnesium, calcium, titanium, iron, cobalt, nickel, copper, zinc and indium, and also zinc(II) salts. Magnesium turnings are pieces of magnesium which are cut to produce small pieces with higher surface areas and generally low amounts of surface oxides (which reduce reactivity). The reactive metal powders of magnesium, calcium, titanium, iron, cobalt, nickel, copper, zinc and indium are Rieke metals, which are prepared by a specific procedure which produces high surface area metal powders which are very reactive in reactions such as those of the present invention. Without wishing to be bound by any particular theory, Rieke metals are thought to be highly reactive because they have high surface areas and lack passivating surface oxides.

As used herein, homogenous refers to compositions which can be described as a single-phase system, as opposed to a heterogeneous system, or multi-phase system. Homogenous also refers to systems which have particles uniformly dispersed throughout the system, or to systems having a uniform composition throughout.

As used herein, agitating refers to the act of shaking, stirring or sonicating a composition, which may be homogenous or heterogeneous, in a container or vessel. In one embodiment, it refers to the act of shaking, stirring or sonicating a composition which may comprise a suspended solid so as to keep the solid from settling in the container or vessel which it occupies.

Disclosed is a process for producing a hydrofluoroalkene, R_{f}CF═CH₂ comprising contacting a hydrofluoroalkanol of structure R_{f}CF₂CH₂OH, with a lewis acid to produce a mixture, diluting said mixture with a solvent to produce a solvent mixture, contacting the solvent mixture with a reactive metal, heating the solvent mixture and reactive metal for a sufficient amount of time to produce a hydrofluoroalkene, and condensing and collecting the volatile products comprising the hydrofluoroalkene. In one embodiment, the product of the hydrofluoroalkanol with a lewis acid becomes homogenous. In one embodiment, the solvent mixture is a solution.

In one embodiment, hydrofluoroalkanols of the formula R_{f}CF₂CH₂OH, such as 1,1,1,2,2-pentafluoro-propanol, an intermediate that may be converted into 2,3,3,3-tetrafluoro-1-propene (HFC-1234yf), are dehydroxyfluorinated. In one embodiment, R_{f} is a perfluoroalkyl group having from one to four carbon atoms. In another embodiment, R_{f} in the above hydrofluoroalkene and hydrofluoroalkanol can be either a Fluorine atom, or a fluorine-substituted alkyl group. In one embodiment, the fluorine-substituted alkyl can be CF₃, C₂F₅, n-C₃F₇, i-C₃F₇, n-C₄F₉, or CHF₂CF₂CF₂—.

In one embodiment, the lewis acid is selected from the group consisting of titanium (IV) halides, zirconium (IV) halides, hafnium (IV) halides, vanadium (III) halides, vanadium (IV) halides, niobium (V) halides, tantalum (V) halides, boron (III) halides and aluminum (III) halides. In one embodiment, the above halides are bromides or chlorides. In another embodiment, the lewis acid is selected from the group consisting of titanium tetrachloride, zirconium tetrachloride, hafnium tetrachloride, vanadium trichloride, vanadium tetrachloride, niobium pentachloride, tantalum pentachloride, boron trichloride, boron trifluoride, aluminum trichloride, aluminum chlorofluoride and aluminum fluoride.

In one embodiment, the contacting step with a lewis acid is conducted for 3 hours. In another embodiment, the contacting step with a lewis acid is conducted for 1 hour. In yet another embodiment, the contacting step with a lewis acid is conducted for about 20 minutes. In yet another embodiment, the contacting step with a lewis acid is conducted until the mixture is homogeneous.

In one embodiment, the product of contacting the hydrofluoroalkanol with a lewis acid is then diluted with a solvent and cooled to produce a solvent mixture. In one embodiment, the solution of the product of contacting the hydrofluoroalkanol with a lewis acid is cooled to from −10° C. +15° C. In one embodiment, the product of contacting the hydrofluoroalkanol with a lewis acid is cooled after dilution with a solvent. In another embodiment, the product of contacting the hydrofluoroalkanol with a lewis acid is cooled concurrently with the dilution with a solvent.

In one embodiment, the solvent is an ether solvent. In another embodiment, the solvent is a polar aprotic solvent selected from the group consisting of dimethylformamide, dimethylsulfoxide, dimethylacetamide, hexamethylphosphoramide, N-methylpyrolidone, tetrahydrofurane, diglyme, ethyleneglycol dimethyl ether, triglyme, 1,4-dioxane, N-methylepyridine, and mixtures thereof. In yet another embodiment, a solvent is selected from the group consisting of diglyme, diethyl ether, tetrahydrofuran, triglyme, 1,4-dioxane, ethylene glycol dimethyl ether, and mixtures thereof.

In one embodiment, the molar ratio of lewis acid to hydrofluoroalkanol is from about 1:1 to about 4:1. In another embodiment, the molar ratio of lewis acid to hydrofluoroalkanol is from about 1:1 to about 3:1. In yet another embodiment, the molar ratio of lewis acid to hydrofluoroalkanol is from about 1:1 to about 2:1.

In one embodiment, the molar ratio of reactive metal to hydrofluoroalkanol is from about 2:1 to about 5:1. In another embodiment, the molar ratio of reactive metal to hydrofluoroalkanol is from about 2:1 to about 4:1. In yet another embodiment, the molar ratio of reactive metal to hydrofluoroalkanol is from about 3:1 to about 4:1.

In one embodiment, the step of contacting the hydrofluoroalkanol with a lewis acid takes place at a temperature of from about −20° C. to about 30° C. In another embodiment, the step of contacting the hydrofluoroalkanol with a lewis acid takes place at a temperature of from about 0° C. to about 20° C. In yet another embodiment, the step of contacting the hydrofluoroalkanol with a lewis acid takes place at a temperature of from about 0° C. to about 10° C. In one embodiment, the contacting step with a lewis acid takes place under a flow of an inert gas, which removes hydrogen chloride formed from the contacting step. In one embodiment, the inert gas is selected from argon or helium. In another embodiment, the inert gas is nitrogen.

In one embodiment the reactive metal is selected from the group consisting of magnesium turnings, activated zinc powder, aluminum, and a powder of any of the following metals: magnesium, calcium, titanium, iron, cobalt, nickel, copper, zinc and indium. In one embodiment, the reactive metal is activated zinc powder.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Group numbers corresponding to columns within the Periodic Table of the elements use the “New Notation” convention as seen in the CRC Handbook of Chemistry and Physics, 81^{st} Edition (2000-2001).

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

### EXAMPLES

The concepts described herein will be further described in the following examples, which do not limit the scope of the invention described in the claims.

### Example 1

Example 1 demonstrates the reduction of octafluoro-1-pentanol to heptafluoro-1-pentene.

A mixture of 6 gm of H(CF₂)₄CH₂OH (26 mM) and 7.4 gm (39 mM) of titanium tetrachloride was stirred until the mixture became homogeneous under an argon flow to remove HCl. Then 40 ml of diglyme were added dropwise while cooling in an ice bath. 6 Gm (91 mM) of zinc powder was added and the mixture was agitated for 3 hours, with the color of the solution getting dark violet. The mixture was heated to 90° C. for 6 hours, and then the volatile products were removed to the cold trap with an argon flow while maintaining the temperature of the solution at 130-140° C. 3.6 Gm of product, HCF₂CF₂CF₂CF═CH₂ and HCF₂CF₂CF₂CF₂CH₃, were obtained in a 2.75:1 ratio.

### Example 2

Example 2 demonstrates the reduction of pentafluoro-1-propanol to 1234yf.

5 Gm of pentafluoropropanol was added to 9.5 gm (50 mM) of titanium tetrachloride, and the mixture was stirred for 40 minutes at 35° C. under argon flow. Then 40 ml of diglyme were added dropwise while cooling in an ice bath. 6.5 Gm (100 Mm) of zinc powder were added, and the mixture was heated at 85° C. for 3 hours. Then the volatile products were removed to a cold trap with an argon purge while maintaining the temperature of the solution at 130-140° C. 2.1 Gm of products were obtained, CF₃CF═CH₂, CF₃CF₂CH₃, and CF₃CH═CH₂, in the following ratio: 60:39:1.

### Example 3

Example 3 demonstrates the reduction of trifluoroethanol to vinylidene fluoride.

5 gm (50 mM) of trifluoroethanol were added to 14.25 gm (75 mM) of titanium tetrachloride, and the mixture was stirred for 40 minutes at 35° C. under an argon flow. Then 50 ml of diglyme were added dropwise under cooling with ice water. 10 gm (150 mM) of zinc powder were added, and the mixture was heated at 85° C. for 4 hours. Then the volatile products were moved to a flask with CCl₄ and 10 gm (62.5 mM) of bromine by slowly bubbling argon through the solution at 130-140° C. The CCl₄ solution was washed with Na₂SO₃ water solution. 1,1-Difluoroethylene was detected in the resulting solution in trace amounts by GCMS and ¹⁹F NMR.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. A process for producing a hydrofluoroalkene, R_{f}CF═CH₂ comprising:
contacting a hydrofluoroalkanol of structure R_{f}CF₂CH₂OH, with a lewis acid to produce a mixture, diluting said mixture with a solvent to produce a solvent mixture, contacting the solvent mixture with a reactive metal, heating the solvent mixture and reactive metal for a sufficient amount of time to produce a hydrofluoroalkene, and condensing and collecting the volatile products comprising the hydrofluoroalkene, wherein R_{f} is F, or a fluorine-substituted alkyl group.

2. The process of claim 1, wherein said fluorine-substituted alkyl group R_{f} is CF₃, C₂F₅, n-C₃F₇, i-C₃F₇, n-C₄F₉, or CHF₂CF₂CF₂—.

3. The process of claim 1, wherein the mole ratio of said lewis acid to hydrofluoroalkanol is from about 1:1 to about 2:1.

4. The process of claim 1, wherein said Lewis acid is selected from the group consisting of titanium (IV) halides, zirconium (IV) halides, hafnium (IV) halides, vanadium (III) halides, vanadium (IV) halides, niobium (V) halides, tantalum (V) halides, boron (III) halides and aluminum (III) halides.

5. The process of claim 1, wherein said Lewis acid is selected from the group consisting of titanium tetrachloride, zirconium tetrachloride, hafnium tetrachloride, vanadium trichloride, vanadium tetrachloride, niobium pentachloride, tantalum pentachloride, boron trichloride, boron trifluoride, aluminum trichloride, aluminum chlorofluoride and aluminum fluoride.

6. The process of claim 1, wherein said solvent is selected from the group consisting of diglyme, diethyl ether, tetrahydrofuran, triglyme, 1,4-dioxane, ethylene glycol dimethyl ether, and mixtures thereof.

7. The process of claim 1, wherein said mixture is cooled to a temperature of from about −10° C. to about 15° C. during the said dilution with solvent.

8. The process of claim 1, wherein said reactive metal is selected from the group consisting of magnesium turnings, activated zinc powder, aluminum, and a powder of any of the following metals:
magnesium, calcium, titanium, iron, cobalt, nickel, copper, zinc and indium.

9. The process of claim 1, wherein the mixture of solvent mixture and reactive metal is heated to a temperature of from 70° C. to 140° C. for from 4 to 6 hours.

10. The process of claim 9, wherein the mixture of solvent mixture and reactive metal is heated to from 80° C. to 95° C.

11. The process of claim 1 wherein the molar ratio of said reactive metal to hydrofluoroalkanol is from about 3:1 to about 4:1.
